(19) United States
(12) Patent Application Publication
(10) Pub. No.: US 2023/0147214 A1
(43) Pub. Date: May 11, 2023

(54) METHODS FOR TREATING AND FOR INHIBITING PROGRESSION OF SEIZURES

(71) Applicant: Allergan, Inc., Irvine, CA (US)

(72) Inventors: Rami Burstein, Chestnut Hill, MA (US); Agustin Melo Carrillo, Boston, MA (US); Mitchell F. Brin, Newport Beach, CA (US)

(21) Appl. No.: 17/820,029

(22) Filed: Aug. 16, 2022

Related U.S. Application Data
(63) Continuation of application No. 16/921,477, filed on Jul. 6, 2020, now abandoned.
(60) Provisional application No. 62/870,872, filed on Jul. 5, 2019.

(51) Int. Cl. A61K 38/48; A61P 25/08; A61K 9/00

(52) U.S. Cl. CPC A61K 38/4893; A61K 9/0019; A61P 25/08

(57) ABSTRACT

Methods for treating seizures and for inhibiting progression of a focal seizure to a generalized seizure by administering extracranially a Clostridial derivative are described.

### CROSS-REFERENCES TO RELATED APPLICATIONS

This patent application is a continuation of U.S. Pat. Application No. 16/921,477 filed Jul. 6, 2020, which claims priority pursuant to 35 U.S.C. § 119(e) to U.S. Provisional Pat. Application No. 62/870,872 filed Jul. 5, 2019, incorporated entirely herein by reference.

### TECHNICAL FIELD

The present disclosure relates to methods for treating seizures and for inhibiting progression of focal seizures to generalized seizures by extracranial administration of a Clostridial derivative.

### BACKGROUND

Seizures are generally associated with abnormal paroxysmal electrical brain activity. The clinical manifestation of seizures may include alterations of consciousness and motor, sensory, autonomic, or psychic events perceived by the patient or an observer. Often, seizures result in temporary loss of consciousness, convulsions, changes in muscle tone, unusual movements and/or staring spells. Seizures can have many causes, such as medicines, high fevers, head injuries, genetic etiologies, infectious diseases, metabolic disorders, stroke, and other diseases. Recurrent unprovoked seizures define the neurologic syndrome known as epilepsy. Seizure can be diagnosed by observation, or by recording of brain electrical activity, as by an electroencephalogram (EEG).

Seizures are divided into three categories based on the symptoms that a patient or an observer perceive, and/or the pattern of electrical brain activity that accompanies a seizure. In broadest terms, seizures can be classified as focal, generalized and unclassified.

Focal (also called “partial”) seizures are those in which the initial semiology or EEG findings indicate onset in only part of one cerebral hemisphere. Notably, focal seizure often arises with asymptomatic abnormal electrical brain activity in only a small volume of cortex, in one hemisphere. Pursuant to their very localized onset, focal seizures spread to adjacent cortical regions within the hemisphere, at which time they are perceived by the patient or observer.

Generalized seizures are those with an initial semiology or EEG findings indicate more than minimal involvement of both cerebral hemispheres and may be subdivided into convulsive seizures and non-convulsive seizures.

Another subcategorization of generalized seizures is into those are primary generalized and arise simultaneously from both hemispheres, and those that are secondarily generalized and arise focally in one hemisphere before spreading to involve the other hemisphere. The third category, unclassified seizures, includes all seizures that defy classification due to incomplete data.

Epilepsy affects 1% of the world population. In individuals with epilepsy experiencing localized (focal or partial) seizures, awareness may be disturbed, and variable degrees of amnesia may be evident, but the seizure does not lead to loss of consciousness. Focal seizures can, however, progress to secondarily generalized seizures, in which case the individual loses consciousness. Clinically, generalized and bilateral seizures are considered more dangerous because of their greater potential for injury and post-seizure complications. Epidemiologically, nearly half of all adults diagnosed with epilepsy experience at least one generalized or bilateral seizure, and about 25% of patients with epilepsy experience them regularly. Current treatments for epilepsy and other seizures include antiepileptic drugs (AED). However, standard-of-care pharmacotherapy is incompletely effective for many patients and can result in unwanted side effects.

### BRIEF SUMMARY

In one aspect, a method for treating seizures associated with a seizure disorder is provided. The method comprises administering to a subject with a seizure disorder a Clostridial derivative in an amount effective for treating seizures, the Clostridial derivative administered extracranially to at least one administration site.

In one embodiment, a method for treating epilepsy is provided. The method comprises administering to a subject with epilepsy a therapeutically effective amount of a Clostridial derivative, the Clostridial derivative administered extracranially to at least one administration site.

In one embodiment, a method for treating aura seizure (or epileptic aura) symptoms is provided. The method comprises administering to a subject with aura seizure (or epilepsy aura) symptoms a therapeutically effective amount of a Clostridial derivative, the Clostridial derivative administered extracranially to at least one administration site.

In one embodiment, the Clostridial derivative is administered by an injection route selected from extramuscular injection, subcutaneous injection, subdermal injection or intradermal injection.

In one embodiment, the Clostridial derivative is administered by injection to the scalp.

In one embodiment, the Clostridial derivative is administered to a suture line within the cervical region of the scalp. In one embodiment, the Clostridial derivative is administered to or in the vicinity of the lambdoid suture, the parietomastoid suture, the occipital suture, or combinations thereof. In an alternative embodiment, the Clostridial derivative is administered to a muscle in the cervical region of the scalp. In another embodiment, the Clostridial derivative is administered to a cervical nerve within the cervical region. In yet another alternative embodiment, the Clostridial derivative is administered to a trigeminal nerve in the cervical region.

In another embodiment, the Clostridial derivative is administered to at least one suture line on the skull of a patient. In one embodiment, the Clostridial derivative is administered to a cervical nerve. In another embodiment, the Clostridial derivative is administered to a trigeminal nerve. In another embodiment, Clostridial derivative is administered to a muscle on the skull of the patient.

In another embodiment, the method comprises administering the Clostridial derivative to more than one administration site. In an exemplary embodiment, the number of administration sites is between 2-8.

In one embodiment, the at least one administration site is approximately along the lambdoid suture.

In another embodiment, the at least one administration site is approximately at the junction of the lambdoid suture and the sagittal suture.

In another embodiment, the at least one administration site is in the vicinity of and/or approximately along any of the suture lines of the skull selected from, for example, frontal suture, squamous suture, coronal suture, lambdoid suture, occipitomastoid suture, parietomastoid suture and sagittal suture.

In an embodiment, the at least one administration site is within an occipital area of the cranium that is innervated by cervical nerves. In a specific embodiment, onabotulinumtoxinA is administered by injection to occipital areas of the cranium that are innervated by cervical nerves.

In an embodiment, the at least one administration site is to any structures or tissues in the vicinity of a trigeminal nerve or cervical nerve, including for example fascia, sub-fascia, aponeurosis, epineurium, or connective tissues in general, muscles, nerves, and suture lines.

In yet other embodiments, the amount of Clostridial derivative administered is evenly divided into portions for administration to each administration site. In other embodiments, a portion of the amount of Clostridial derivative is administered at each administration site, where the portion administered at each administration site is unequal.

In other embodiments, the Clostridial derivative is a botulinum neurotoxin. In one embodiment, the botulinum neurotoxin is botulinum toxin type A, botulinum toxin type B, botulinum toxin type C, botulinum toxin type D, or botulinum toxin type E.

In one embodiment, the botulinum toxin is a botulinum toxin type A. In some embodiments, the botulinum toxin type A is selected from the group consisting of onabotulinumtoxinA, incobotulinumtoxinA, abobutulinumtoxinA, daxibotulinumtoxinA, prabotulinumtoxinA, and combinations thereof. In some embodiments, the amount of botulinum neurotoxin type A administered is between about 1 Units and 3000 Units. In some embodiments, the amount of botulinum neurotoxin type A administered is between 2 Units and 200 Units. In some other embodiments, the amount of botulinum neurotoxin type A administered is between 200 Units and 500 Units. In yet other embodiments, the amount of botulinum neurotoxin administered is greater than 500 Units.

In one embodiment, the botulinum toxin comprises a botulinum toxin complex. In another embodiment, the botulinum toxin comprises a pure neurotoxin.

In one embodiment, the botulinum toxin is onabotulinumtoxinA and is administered in an amount of between about 2-200 Units. In one embodiment, the amount administered is evenly divided among about 2-10 administration sites.

In still other embodiments, the seizure is selected from a focal seizure, a myoclonic seizure, and a generalized tonic-clonic seizure.

In another embodiment, the seizures are associated with a seizure disorder selected from epilepsy, juvenile myoclonic epilepsy, idiopathic generalized epilepsy, and Lennox-Gastaut syndrome.

In other embodiments, the seizure is a generalized tonic-clonic seizure associated with Lennox-Gastaut syndrome.

In one embodiment, the step of administering the Clostridial derivative is an adjunctive therapy of an oral, subcutaneous, rectal or intravenous medication given to treat seizures.

In another embodiment, the step of administering the Clostridial derivative is an adjunctive therapy of electrical stimulation of the vagus nerve, or facial nerve, or thalamus, or cerebral cortex that is prescribed to treat seizures.

In another aspect, a method for inhibiting progression of a focal seizure to a bilateral seizure is provided. The method comprises administering extracranially to a subject afflicted by focal seizures a Clostridial derivative in a therapeutically effective amount to inhibit propagation of the abnormal brain electrical activity to nearby brain areas, thereby preventing spread and/or generalization of a focal seizure.

In another aspect, a method for preventing progression of a focal asymptomatic seizure to a focal symptomatic seizure is provided. The method comprises administering extracranially to a subject afflicted by focal seizures a Clostridial derivative in a therapeutically effective amount to inhibit propagation of the abnormal brain electrical activity to nearby brain areas, thereby preventing progression of a focal asymptomatic seizure to a focal symptomatic seizure.

In yet another aspect, a method for treating seizures associated with a seizure disorder is provided which comprises administering extracranially to a subject afflicted by seizures a Clostridial derivative in a therapeutically effective amount for treating the seizures.

In still another aspect, a method for treating seizures in a subject with epilepsy is provided which comprises administering extracranially to the subject a therapeutically effective amount of a Clostridial derivative for treating the seizures.

In still another aspect, a method for controlling focal epileptic seizures comprises administering extracranially to a subject a Clostridial derivative in an amount effective to therapeutically effective amount to control the focal epileptic seizure.

In one embodiment, the Clostridial derivative is administered to the scalp.

In some embodiments, the methods in accordance with the present disclosure further comprise administering to the subject an adjunctive therapy for seizure. In some embodiments, the adjunctive therapy comprises one or more anti-epileptic drugs.

In still another aspect, a method for treating ictal headaches is provided which comprises administering extracranially to the subject a therapeutically effective amount of a Clostridial derivative for treating the ictal headaches. In one embodiment, the Clostridial derivative is administered before a seizure (pre-ictal). In another embodiment, the Clostridial derivative is administered after a seizure (post-ictal).

### BRIEF DESCRIPTION OF THE DRAWINGS

The following drawings are presented to illustrate aspects and features of embodiments of the present methods.

FIG. 1A is a schematic top view of a test animal showing certain cranial suture lines and administration sites for delivery of a Clostridial derivative, in one embodiment;

FIG. 1B is a top view schematic of the cortical anatomy showing the location of picrotoxin administration used to trigger seizure and the location of glass pipette electrodes used for recording electrocorticograms in the different cortical areas;

FIGS. 2A-2B are electrocorticogram traces of a test animal occipital cortex (FIG. 2A) and parietal cortex (FIG. 2B) prior to and after topical application of picrotoxin to the occipital cortex to trigger a focal seizure, the time of picrotoxin application indicated by the dotted line in FIG. 2A;

FIGS. 3A-3B are electrocorticogram traces of a test animal occipital cortex (FIG. 3A) and parietal cortex (FIG. 3B), the animal previously treated with an exemplary Clostridial derivative (onabotulinumtoxinA) prior to induction of a seizure with picrotoxin (given at the time indicated by the dotted line in FIG. 3A);

FIGS. 4A-4B are diagrams of a human skull (FIG. 4A) and a rat skull (FIG. 4B) showing the absence of trigeminal nerves in the cervical region where the Clostridial derivative was administered; and

FIGS. 5A, 5B and 5C are diagrams of a human skull showing various suture lines.

### DETAILED DESCRIPTION

### Definitions

The following definitions apply herein:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a pharmaceutical composition to a subject, or alternatively a subject receiving a pharmaceutical composition.

“Alleviating” means a reduction in the occurrence of seizures associated with a condition or disorder. Thus, alleviating includes some reduction, significant reduction, near total reduction, and total reduction.

“Biological activity” describes the beneficial or adverse effects of a drug on living matter. When a drug is a complex chemical mixture, this activity is exerted by the substance’s active ingredient but can be modified by the other constituents. Biological activity can be assessed as potency or as toxicity by an in vivo LD₅₀ or ED₅₀ assay, or through an in vitro assay such as, for example, cell-based potency assays as described in U.S. 20100203559 and U.S. 20100233802.

“Botulinum toxin” means a neurotoxin produced by Clostridium botulinum, as well as a botulinum toxin, fragments, variants or chimeras thereof made recombinantly by a non-Clostridial species. The term “botulinum toxin”, as used herein, encompasses Botulinum toxin serotype A (BoNT/A), Botulinum toxin serotype B (BoNT/B), Botulinum toxin serotype C (BoNT/C), Botulinum toxin serotype D (BoNT/D), Botulinum toxin serotype E (BoNT/E), Botulinum toxin serotype F (BoNT/F), Botulinum toxin serotype G (BoNT/G), Botulinum toxin serotype H (BoNT/H), Botulinum toxin serotype X (BoNT/X), Botulinum toxin serotype En (BoNT/En), and mosaic Botulinum toxins and/or their subtypes and any other types of subtypes thereof, or any re-engineered proteins, analogs, derivatives, homologs, parts, sub-parts, variants, or versions, in each case, of any of the foregoing. “Botulinum toxin”, as used herein, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins.

“Clostridial derivative” refers to a molecule which contains any part of a clostridial toxin as defined herein. As used herein, the term “clostridial derivative” encompasses native or recombinant neurotoxins, recombinant modified toxins, fragments, chimeras and variants thereof, a Targeted Vesicular Exocytosis Modulator (TEM), or combinations thereof.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin like BoNT/A, a BoNT/B, a BoNT/Ci, a BoNT/D, a BoNT/E, a BoNT/F, a BoNT/G, a BoNT/H, a BoNT/X, a BoNT/En, mosaic Botulinum toxins, a Tetanus toxin (TeNT), a Baratii toxin (BaNT), and a Butyricum toxin (BuNT). The BoNT/C₂ cytotoxin and BoNT/C₃ cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof. A Clostridial toxin disclosed herein also includes a Clostridial toxin complex as well as the neurotoxic component of the clostridial toxin that is unassociated with the non-toxin associated proteins (NAPs). As used herein, the term “Clostridial toxin complex” refers to a complex comprising the neurotoxic component of the Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

“Clostridial toxin active ingredient” refers to a molecule which contains any part of a clostridial toxin that exerts an effect upon or after administration to a subject or patient. As used herein, the term “clostridial toxin active ingredient” encompasses a Clostridial toxin complex comprising the approximately 150-kDa Clostridial toxin and other proteins collectively called non-toxin associated proteins (NAPs), the approximately 150-kDa Clostridial toxin alone, or a modified Clostridial toxin, such as, e.g., a re-targeted Clostridial toxins.

“Effective amount” as applied to the biologically active ingredient means that amount of the ingredient which is generally sufficient to effect a desired change in the subject. For example, where the desired effect is alleviating seizures or inhibiting seizure progression, an effective amount of the ingredient is that amount which causes at least a substantial reduction in frequency of seizures or in the likelihood of a focal seizure progressing to a generalized seizure, and without resulting in significant toxicity. The effective amount of a clostridial derivative can be measured in potency or toxicity unit (e.g. LD₅₀ or ED₅₀) or in mass units (e.g. in ng or mg).

“Extracranial administration” means administration to a site external to the cranium. In some embodiments, extracranial administration refers to administration to any suture line, or combination of suture lines on the skull of a patient.

“Local administration” means administration of a pharmaceutical agent at or to the vicinity of a site on or within an animal body, at which site a biological effect of the pharmaceutical is desired, such as via, for example, intramuscular or intra- or subdermal injection or topical administration. Local administration excludes systemic routes of administration, such as intravenous or oral administration. Topical administration is a type of local administration in which a pharmaceutical agent is applied to a patient’s skin.

“Modified botulinum toxin” means a botulinum toxin that has had at least one of its amino acids deleted, modified, or replaced, as compared to a native botulinum toxin. Additionally, the modified botulinum toxin can be a recombinantly produced neurotoxin, or a derivative or fragment of a recombinantly made neurotoxin. A modified botulinum toxin retains at least one biological activity of the native botulinum toxin, such as, the ability to bind to a botulinum toxin receptor, or the ability to inhibit neurotransmitter release from a neuron. One example of a modified botulinum toxin is a botulinum toxin that has a light chain from one botulinum toxin serotype (such as serotype A), and a heavy chain from a different botulinum toxin serotype (such as serotype B). Another example of a modified botulinum toxin is a botulinum toxin coupled to a neurotransmitter, such as substance P.

“Mutation” means a structural modification of a naturally occurring protein or nucleic acid sequence. For example, in the case of nucleic acid mutations, a mutation can be a deletion, addition or substitution of one or more nucleotides in the DNA sequence. In the case of a protein sequence mutation, the mutation can be a deletion, addition or substitution of one or more amino acids in a protein sequence. For example, a specific amino acid comprising a protein sequence can be substituted for another amino acid, for example, an amino acid selected from a group which includes the amino acids alanine, asparagine, cysteine, aspartic acid, glutamic acid, phenylalanine, glycine, histidine, isoleucine, lysine, leucine, methionine, proline, glutamine, arginine, serine, threonine, valine, tryptophan, tyrosine or any other natural or non-naturally occurring amino acid or chemically modified amino acids. Mutations to a protein sequence can be the result of mutations to DNA sequences that when transcribed, and the resulting mRNA translated, produce the mutated protein sequence. Mutations to a protein sequence can also be created by fusing a peptide sequence containing the desired mutation to a desired protein sequence.

“Patient” means a human or non-human subject receiving medical or veterinary care. Accordingly, the compositions as disclosed herein can be used in treating any animal, such as, for example, mammals, or the like.

“Peripherally administering” or “peripheral administration” means subdermal, intradermal, transdermal, subcutaneous administration, or intramuscular administration.

“Pharmaceutical composition” means a composition comprising an active pharmaceutical ingredient, such as, for example, a clostridial toxin such as a botulinum toxin, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“TEMs”, abbreviated for Targeted Exocytosis Modulators are retargeted endopeptidases that direct the catalytic activity of the light chain to specific types of neuronal cells or to target cells that were not affected by botulinum toxins expanding the beneficial clinical effect of inhibition of exocytosis in several human diseases.

“Treating” or “treatment” means to alleviate (or to eliminate) at least one symptom, either temporarily or permanently. More particularly, as used herein “treatment” of seizures, of a seizure disorder or symptoms thereof includes one or more of reducing the frequency (or to prevent) or reducing the duration of seizures, the overall disorder or symptoms thereof, ameliorating the severity of the seizures, the overall disorder, or symptoms thereof, treating seizures, the overall disorder or symptoms thereof, curing the seizures, the overall disorder or symptoms thereof, achieving a reduction in the number or severity of the seizures or symptoms thereof, improving the quality of life of the patient experiencing the seizures.

“Variant” means a clostridial neurotoxin, such as wild-type botulinum toxin serotype A, B, C, D, E, F, G, H, X, or mosaic botulinum toxins that has been modified by the replacement, modification, addition or deletion of at least one amino acid relative to wild-type botulinum toxin, which is recognized by a target cell, internalized by the target cell, and catalytically cleaves a SNARE (SNAP (Soluble NSF Attachment Protein) Receptor) protein in the target cell. An example of a variant neurotoxin component can comprise a variant light chain of a botulinum toxin having one or more amino acids substituted, modified, deleted and/or added. This variant light chain may have the same or better ability to prevent exocytosis, for example, the release of neurotransmitter vesicles. Additionally, the biological effect of a variant may be decreased compared to the parent chemical entity. For example, a variant light chain of a botulinum toxin type A having an amino acid sequence removed may have a shorter biological persistence than that of the parent (or native) botulinum toxin type A light chain.

As used herein the term “improving the seizure disorder” or “improving seizures” intends reducing the frequency of seizures and/or associated symptoms, and/or reducing the severity of the seizures (stabilization of the seizure state so that it does not progress from, for example, focal to generalized) and/or associated symptoms, and/or maintaining an essentially seizure free state in a subject, and/or ameliorating undesired seizures associated with a disease or disorder, or a combination of any of the above.

By “seizures” it is meant any non-chronic or chronic seizure, inclusive of, seizures resulting from epilepsy, epilepsy disorder, audiogenic seizure disorder, epilepsy syndromes and related conditions. Seizures resulting from fever, abnormal blood levels of sodium or glucose, drug abuse, certain medications, high blood pressure, phenylketonuria and uremia, as well as acute seizure disorders such as those caused by a brain tumor and/or brain injury, which may in turn become chronic seizure disorders. The term “epileptic disorder” or “epilepsy” refers to a chronic neurological syndrome characterized by recurrent seizures.

The term “epilepsies” refers to those conditions involving chronic recurrent epileptic seizures that can be considered epileptic disorders. The term epileptic seizure should be understood to encompass any state of seizure whether a part of the epileptic disorder or as a result of another syndrome or related condition. Non-limiting examples for types of epileptic seizures are focal seizures (also called simple partial seizures, complex partial seizures, partial seizures with secondarily generalization seizures, partial elementary seizures, complex partial seizures, or psychomotor seizures), generalized seizures (convulsive and nonconvulsive; e.g. absence seizures, atypical absence seizures, myoclonic seizures, clonic seizures, tonic seizures, tonic-clonic seizures, atonic seizures (astatic), secondary generalized seizures and other chronic unclassifiable seizures.

As used herein, the term “partial seizure” refers to a seizure affecting a limited area of the brain. In contrast, a “generalized seizure” is a seizure affecting the entire brain. Symptoms of partial seizures include, but are not limited to, abnormal muscle contraction, staring spells, forced eye movements, abnormal sensations such as numbness and tingling, hallucinations, abdominal pain, nausea, abnormal sweating, rapid heart rate, vision changes, mood changes, and loss of consciousness. Partial seizures are generally classified as “simple” or “complex,” based on observed symptoms, analysis of electroencephalographic (EEG) data, or other factors.

As used herein, the term “complex partial seizure” refers to a partial seizure that causes a partial or full loss of consciousness. Complex partial seizures can occur in any area of the brain. Complex partial seizures frequently occur in one of the brain’s two temporal lobes. Typical complex partial seizures last for one to two minutes and can be preceded by an “aura” (i.e., a warning sensation such as nausea) that is indicative of an oncoming seizure.

As used herein, the term “aura’, “aura seizure symptoms” or “epileptic aura symptoms” refer to any warning sensation perceived by a patient that is indicative of an oncoming seizure or epilepsy, which may include, for example, nausea, visual changes, auditory changes, olfactory changes, perception of a strange light, tearfulness of the eyes, an unpleasant smell (phantosmia) or tastes (gustatory hallucinations), synesthesia, cephalic aura, confusing thoughts or experiences, sudden feeling of anxiety, fear or vertigo, numbness or tingling sensation, and hallucinations, etc.

As used herein, the term “simple partial seizure” refers to a partial seizure that does not cause a loss of consciousness. Typical simple partial seizures last 60 seconds or less.

As used herein, the term “limbic seizure” refers to a seizure originating in one of the brain’s limbic structures. The limbic system contributes to regulation of the body’s unconscious movement and hormonal activity. Limbic structures include, but are not limited to, the thalamus, hypothalamus, cingulate gyrus, amygdala, hippocampus, and basal ganglia.

As used herein, the term “chemically-induced seizure” refers to a seizure in a subject resulting from exposure of the subject to a chemical substance.

As used herein, the term “therapeutically effective amount” refers to an amount sufficient to achieve a desired therapeutic effect.

As used herein, the term “in the vicinity of” refers to is at or within about 1.5 cm, more preferably at or within about 1.0 cm and most preferably at or within about 0.5 cm of a specified referenced location, e.g. when botulinum toxin is administered in accordance with the present disclosure, it is administered within the vicinity of at least one suture line of the patient, i.e. at or within about 1.5 cm, more preferably at or within about 1.0 cm or most preferably at or within 0.5 cm away from the referenced suture line.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### Methods of Use

In one aspect, methods for treating seizures associated with a seizure disorder and for inhibiting progression of a focal seizure to a generalized seizure, methods for treating aura seizure (or epileptic aura) symptoms, or methods for treating ictal-headaches are provided. The methods comprise administering extracranially to a subject a Clostridial derivative or Clostridial toxin in a therapeutically effective amount. A study showing that extracranial administration of a Clostridial derivative treated seizures in animals by inhibiting progression of a focal seizure to a generalized seizure was conducted and is described in Example 1. In this study, a first group of animals was treated with a Clostridial derivative prior to chemically inducing a seizure. A second group of animals was treated with saline and a third group of animals received no prior treatment, before induction of a chemical seizure.

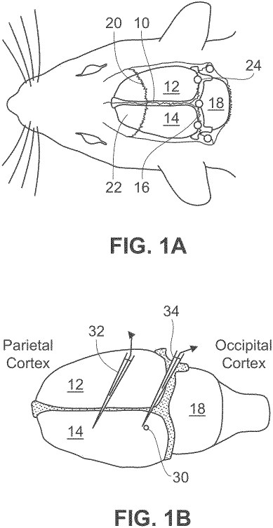

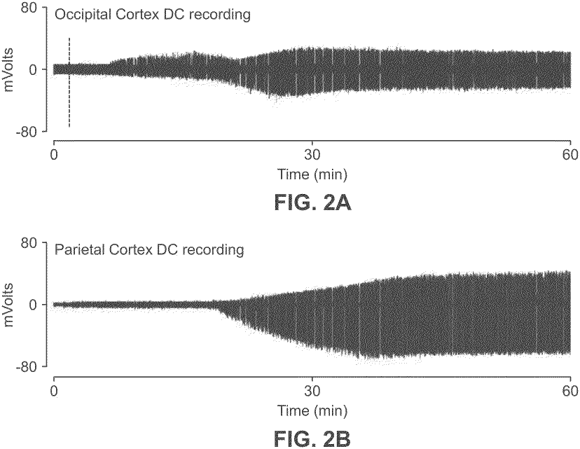

With regard to the animals treated with a Clostridial derivative prior to chemically inducing a seizure, FIG. 1A is a schematic illustration in top view of an animal’s cranium with a cut away to show location of certain cranial suture lines. The sagittal suture 10 is the connective tissue joint between the two parietal bones, 12, 14. The lambdoid suture 16 is the connective tissue joint on the posterior aspect of the skull that connects the parietal bones with the occipital bone 18. The coronal suture 20 is the connective tissue joint that separates the frontal bone 22 and the parietal bones 12 and 14. In the study described in Example 1, a Clostridial derivative was administered on or along the lambdoid suture. The Clostridial derivative dose was equally divided into portions, in this study into five portions, and a portion was administered by injection to five administration sites along the lambdoid suture. The five administration sites are identified in FIG. 1A by dots, such as dot 24, which is representative. The animals in the test group treated with a Clostridial derivative prior to a seizure received the Clostridial derivative dose between 7-12 days prior to induction of a chemical seizure with picrotoxin. The animals in the saline test group were similarly treated except each animal received saline at the administration site instead of Clostridial derivative.

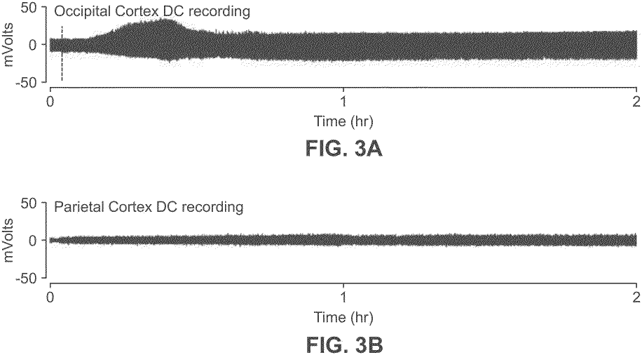

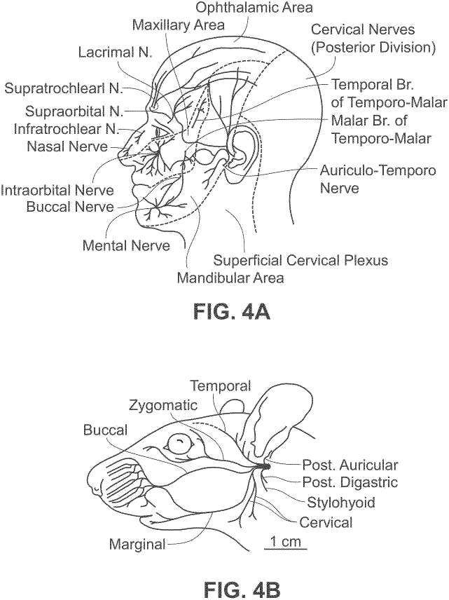

To induce a seizure, the picrotoxin was administered topically at the location identified by numerical identifier 30 in FIG. 1B. Electrodes 32, 34 for recording local field potential and electrocorticogram were placed in the parietal cortex and the occipital cortex of all test animals. The electrocorticogram traces are shown in FIGS. 2-3. In FIG. 2A the trace from the occipital cortex as a function of time, in minutes, is shown for the saline treated animal group. The trace from the parietal cortex for the saline-treated animals is shown in FIG. 2B. Picrotoxin was administered just after time zero, the administration time indicted by the dotted line in FIG. 2A. Several minutes after picrotoxin administration to the occipital cortex, a focal seizure is induced in the occipital cortex (FIG. 2A) which then progresses to the parietal cortex (FIG. 2B). The traces in FIGS. 2A-2B show that in untreated animals, induction of a seizure with picrotoxin triggers a focal seizure that progresses to a generalized seizure.

FIG. 3A shows the trace from the occipital cortex for an animal in the group treated with a Clostridial toxin prior to the picrotoxin-induced seizure. Picrotoxin was administered just after time zero, the time of administration indicted by the dotted line in FIG. 3A. Several minutes after picrotoxin administration to the occipital cortex, a focal seizure is induced in the occipital cortex (FIG. 3A). However, as seen in FIG. 3B, the focal seizure does not progress to a generalized seizure. These observations suggest that administration of a Clostridial derivative inhibited or essentially prevented progression of a focal seizure to a generalized seizure.

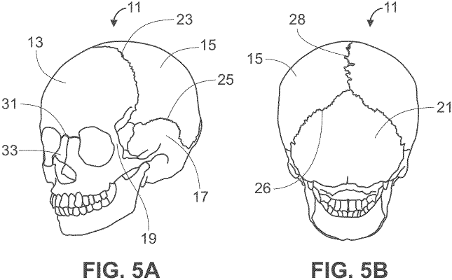

In the study described above and in Example 1, the Clostridial derivative was administered extracranially along the lambdoid suture. FIGS. 4A-4B are diagrams of a human skull (FIG. 4A) and a rat skull (FIG. 4B) showing the absence of trigeminal nerves in the cervical region where the Clostridial derivative was administered. Thus, in one embodiment, administering the Clostridial derivative and/or each administration site excludes a trigeminal nerve. The extracranial administration was done by injection into the scalp, which does not include any muscles. The scalp is a layered structure, the outermost layer being skin (with sebaeceous glands and hair follicles), followed by connective tissue, the epicranial aponeurosis, the loose areolar connective tissue, and the pericranium. In one embodiment, the Clostridial derivative is administered by injection into all layers of the scalp, and in other embodiments it is administered into one, two, three, four, and/or five layers of the scalp.

In one embodiment, the Clostridial derivative is administered to a plurality of administration sites that include the scalp and/or the cervical region. For example, the Clostridial derivative can be administered to a plurality of administration sites in the occipitalis muscle and a plurality of administration sites in the cervical paraspinal muscle and/or the scalp. In a more specific example, the Clostridial derivative is administered to between about 1-10, 1-8, 1-6, 2-6, 2-5 or 2-4 sites in the occipitalis muscle, and/or 2-6, 2-4 sites in the cervical paraspinal muscle, and/or 1-8, 1-6, 2-6, 3-5 sites in the scalp, which may include at or along a suture or a suture junction. The administration can be by injection, for example, intramuscularly, intradermally, and/or subcutaneously. The administration desirably, in some embodiments, delivers the Clostridial derivative to the cervical nerve. In one embodiment, at least one administration site is to the occipital areas of the cranium that are innervated by cervical nerves.

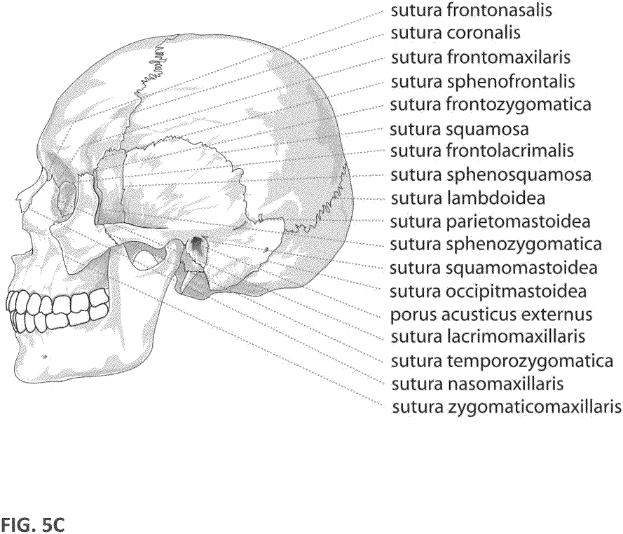

In one embodiment, at least one administration site is in the vicinity and/ or approximately along any of the suture lines of the skull as exemplified in FIGS. 5A, 5B and 5C. FIG. 5A is a perspective front view and FIG. 5B is a rear view of a human skull 11. FIG. 5C also shows some less common suture lines which have been identified on a human skull. The bony plates that comprise the skull include the frontal bone 13, the parietal bone 15, the temporal bone 17, sphenoid bone 19 and the occipital bone 21. The edges at which these bony plates meet are held together by cranial sutures. For example, the coronal suture 23 (also referred to as coronalis in FIG. 5C) is at the junction of the frontal 13 and parietal 15 bones. The squamous suture 25 (also referred to as squamosa in FIG. 5C) is at the junction between the parietal 15 and temporal 17 bones. The lambdoid suture 26 (also referred to as lambdoidea in FIG. 5C) is at the junction between the parietal 15 and occipital 21 bones. The sagittal suture 28 (not shown in FIG. 5C) is at the junction between two parietal bones, and the frontonasal suture 31 is at the junction between the frontal bone 13 and the nasal bone 33.

Accordingly, a method for treating seizure and/or for inhibiting or preventing progression of a seizure and/or inhibiting or preventing secondary spread from a seizure focus is contemplated. The method comprises administering a Clostridial derivative to a subject in need. In one embodiment, the Clostridial derivative is administered extracranially. In some embodiments, it is administered extracranially to the scalp. In some embodiments, it is administered to at least one suture line in the skull. In other embodiments, it is administered to a trigeminal nerve in the skull. In yet other embodiments, it is administered to a muscle in the skull.

In some embodiments, it is administered to at least one suture line in the cervical region. In other embodiments, it is administered to a trigeminal nerve in the cervical region. In yet other embodiments, it is administered to a muscle in the cervical region.

In other embodiments, the Clostridial derivative is administered by extramuscular injection, subcutaneous injection, subdermal injection or intradermal injection. The Clostridial derivative may be administered to one site or to more than one site or to a plurality of sites. In one embodiment, the Clostridial derivative is administered to between about 2-15 sites, between about 2-12 sites, between about 2-10 sites, between about 2-8 sites, between about 2-6 sites, between about 2-5 sites, between about 3-6 sites, between about 3-5 sites, or to 3, 4, 5, 6, 7, 8, 9, or 10 sites. In one embodiment, the sites are injection sites.

As mentioned, the Clostridial derivative is administered extracranially. It is contemplated to administer to one or more sites on or adjacent a cranial suture, such as the lambdoid suture, the sagittal suture, the coronal suture, the squamosal suture. The Clostridial derivative can be administered to a suture and/or a junction of sutures, such as the junction of the lambdoidal and sagittal sutures, or the junction of the coronal and sagittal suture. In one embodiment, the Clostridial derivative is administered to the occipital area of the cranium. Within the occipital area, the administration may be to an area that is innervated by cervical nerves.

In some embodiments, the Clostridial derivative administration is lateralized to the primary region of the seizure. In another embodiment, the Clostridial derivative is administered on the opposite side of the primary region of the seizure.

In some embodiments, the Clostridial derivative is a botulinum toxin type A. In some embodiments, the Clostridial derivative is selected from onabotulinumtoxinA, abobotulinumtoxinA, incobotulinumtoxinA, daxibotulinumtoxinA, prabotulinumtoxinA, and combinations thereof. In one embodiment, botulinum toxin type A may be administered to the sagittal suture line at about 5-50 units divided among 2-6 sites; to the coronal suture line at about 10-60 units divided among 2-8 sites; to the temporalis muscle and/or the squamosal suture at about 10-100 units divided among 2-20 sites; to the occipitalis and/or the lambdoid suture at about 10-60 units divided among 2-10 sites; and to at least one of the cervical paraspinal at about 1-20 units divided at one site or divided among 2-4 sites, the corrugator at about 1-30 units divided among 2-6 sites and the procerus at about 1-10 units at 1-4 sites. In one embodiment, the treatment paradigm further comprises administering botulinum toxin type A at a dose of about 5 units at 1 site to a lambdoid-sagittal junction.

Other examples include administering botulinum toxin type A to the sagittal suture line at about 15 units divided among 3 sites; to the coronal suture line at about 50 units divided among 5 sites; to the squamosal suture at about 30 units divided among 6 sites; to the lambdoidal suture at about 30 units divided among 6 sites; and/or to the lambdoid-sagittal junction at about 1 unit at 1 site.

In one embodiment, the seizures experienced by the subject to be treated are associated with a disorder or a condition. Non-limiting examples include epilepsy, juvenile myoclonic epilepsy, idiopathic generalized epilepsy, and Lennox-Gastaut syndrome. In another embodiment, the seizures experienced by the subject to be treated are associated with an epilepsy syndrome, including epilepsy, or other disorder characterized by seizures. Non-limiting examples of epilepsy syndromes and other disorders associated with seizures include benign familial neonatal seizures, early myoclonic encephalopathy, Ohtahara syndrome, migrating partial seizures of infancy, West syndrome, benign myoclonic epilepsy in infancy, benign familial infantile seizures, benign infantile seizures (nonfamilial), Dravet syndrome, hemiconvulsion-hemiplegia syndrome, benign childhood epilepsy with centrotemporal spikes, early-onset benign childhood occipital epilepsy (Panayiotopoulos type), late-onset childhood occipital epilepsy (Gastaut type), epilepsy with myoclonic absences, epilepsy with myoclonicastatic seizures, Lennox-Gastaut syndrome, Landau-Kleffner syndrome (LKS), epilepsy with continuous spike-and-waves during slow-wave sleep (other than LKS), childhood absence epilepsy, progressive myoclonus epilepsies, idiopathic generalized epilepsies with variable phenotypes, juvenile absence epilepsy, juvenile myoclonic epilepsy, epilepsy with generalized tonic-clonic seizures only, reflex epilepsies, idiopathic photosensitive occipital lobe epilepsy, startle epilepsy, autosomal dominant nocturnal frontal lobe epilepsy, familial temporal lobe epilepsies, generalized epilepsies with febrile seizures plus, familial focal epilepsy with variable foci, symptomatic focal epilepsies, limbic epilepsies, mesial temporal lobe epilepsy with hippocampal sclerosis, Mesial temporal lobe epilepsy defined by specific etiologies.

Use of the methods herein are also suitable for treating seizures and/or inhibiting progression of seizures in patients experiencing seizures not associated with epilepsy. Conditions with seizures that do not require a diagnosis of epilepsy include, for example, benign neonatal seizures, febrile seizures, reflex seizures, alcohol-withdrawal seizures, drug or other chemically induced seizures, immediate and early post-traumatic seizures, single seizures or isolated clusters of seizures, rarely repeated seizures (oligoepilepsy). In one embodiment, the seizure to be treated is a partial onset seizure, a myoclonic seizure, or a primary generalized tonic-clonic seizure. In another embodiment, the seizure is a generalized tonic-clonic seizure associated with Lennox-Gastaut syndrome.

The methods described herein may be an adjunctive therapy of an oral, subcutaneous, rectal or intravenous medication given to stop or prevent seizures, which are well known in the art.

### Clostridial Derivatives

In one embodiment, the Clostridial derivative is a botulinum neurotoxin (BoNT), such as, for example, BoNT/A, BoNT/B, etc. These toxins act on the nervous system by blocking the release of neurosecretory substances such as neurotransmitters. The action of BoNT is initiated by its binding to a receptor molecule on the cell surface, and then the toxin-receptor complex undergoes endocytosis. Once inside the cell, BoNT cleaves exocytotic specific proteins responsible for neurotransmitter docking and release from the cell known as the SNARE proteins (soluble N-ethylmaleimide-sensitive factor attachment protein receptor). The resulting transient chemodenervation has been utilized medically to block motor neurotransmission at the neuromuscular junction leading to a variety of therapeutic applications.

In some embodiments, the clostridial derivative includes a native, recombinant clostridial toxin, recombinant modified toxin, fragments thereof, TEMs, or combinations thereof. In some embodiments, the clostridial derivative is a botulinum toxin. In some embodiments, the botulinum toxin can be a botulinum toxin type A, type B, type Ci, type D, type E, type F, or type G, type H, type X, mosaic botulinum toxins or any combination thereof. The botulinum neurotoxin can be a recombinantly made botulinum neurotoxin, such as botulinum toxins produced by E. coli. In alternative embodiments, the Clostridial derivative is a TEM.

In some embodiments, the botulinum neurotoxin can be a modified neurotoxin, that is a botulinum neurotoxin which has at least one of its amino acids deleted, modified or replaced, as compared to a native toxin, or the modified botulinum neurotoxin can be a recombinant produced botulinum neurotoxin or a derivative or fragment thereof. In certain embodiments, the modified toxin has an altered cell targeting capability for a neuronal or non-neuronal cell of interest. This altered capability is achieved by replacing the naturally-occurring targeting domain of a botulinum toxin with a targeting domain showing a selective binding activity for a non-botulinum toxin receptor present in a non-botulinum toxin target cell. Such modifications to a targeting domain result in a modified toxin that is able to selectively bind to a non-botulinum toxin receptor (target receptor) present on a non-botulinum toxin target cell (retargeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

The Clostridial derivative, such as a botulinum toxin, for use according to the present methods can be stored in lyophilized, vacuum dried form in containers under vacuum pressure or as stable liquids. Prior to lyophilization the botulinum toxin can be combined with pharmaceutically acceptable excipients, stabilizers and/or carriers, such as, for example, albumin, or the like. In embodiments containing albumin, the albumin can be, for example, human serum albumin, or the like. The lyophilized material can be reconstituted with a suitable liquid such as, for example, saline, water, or the like to create a solution or composition containing the botulinum toxin to be administered to the patient.

In some embodiments, the Clostridial derivative is provided in a controlled release system comprising a polymeric matrix encapsulating the clostridial derivative, wherein a fractional amount of the Clostridial derivative is released from the polymeric matrix over a prolonged period of time in a controlled manner. Controlled release neurotoxin systems have been disclosed for example in U.S. Pat. Nos. 6,585,993; 6,585,993; 6,306,423 and 6,312,708, each of which is hereby incorporated by reference in its entirety.

In alternative embodiments, the Clostridial derivative is provided in an ointment, gel, cream, or emulsion suitable for topical administration.

The therapeutically effective amount of the Clostridial derivative, for example a botulinum toxin, administered according to the present methods can vary according to the potency of the toxin and particular characteristics of the pain being treated, including its severity and other various patient variables including size, weight, age, and responsiveness to therapy. The potency of the toxin is expressed as a multiple of the LD₅₀ value for the mouse, one unit (U) of toxin being defined as being the equivalent amount of toxin that kills 50% of a group of 18 to 20 female Swiss-Webster mice, weighing about 20 grams each. The potency of the clostridial derivative can also be expressed in mass units. The effective amount in mass units can be determined based on the intended effect. For example, the effective amount can be determined based on the amount of the clostridial derivative required to alleviate seizures or inhibit seizure progression from a focal seizure to a generalized seizure, and without resulting in significant toxicity.

The therapeutically effective amount of the botulinum toxin can vary according to the potency of a particular botulinum toxin, as commercially available Botulinum toxin formulations do not have equivalent potency units. It has been reported that one Unit of BOTOX® (onabotulinumtoxinA), a botulinum toxin type A available from Allergan, Inc., has a potency Unit that is approximately equal to 3 to 5 Units of DYSPORT® (abobotulinumtoxinA), also a botulinum toxin type A available from Ipsen Pharmaceuticals. MYOBLOC®, (known as NEUROBLOC® outside the United States), a botulinum toxin type B available from Elan, currently USWorldmeds, has been reported to have a much lower potency Unit relative to BOTOX®. In some embodiments, the botulinum neurotoxin can be a pure toxin, devoid of complexing proteins, such as XEOMIN® (incobotulinumtoxinA). Thus, the quantity of toxin administered and the frequency of its administration will be at the discretion of the physician responsible for the treatment and will be commensurate with questions of safety and the effects produced by a particular toxin formulation. In embodiments, the Clostridial derivative is selected from onabotulinumtoxinA, incobotulinumtoxinA, abotulinumtoxinA, daxibotulinumtoxinA, prabotulinumtoxinA, and rimabotulinumtoxinB.

The dose of Clostridial derivative to be administered can be evenly divided into portions for administration to each administration site where more than one administration site is desired. It is also contemplated that the dose to be administered is divided into unequal portions for administration at each administration site.

In one embodiment, treatment effects of the Clostridial derivative persist for between about 1 month and 5 years, or for between about 1 month and 1 year, or for between about 1 month and 6 months, or for between about 1 month and 5 months, for between about 1 month and 4 months, for between about 1 month and 3 months, for between about 1 month and 2 months. In some embodiments, the treatment can be provided continuously or intermittently as needed.

In one embodiment, the Clostridial derivative is in a composition that is a stable liquid or solid pharmaceutical composition. An exemplary composition comprises the Clostridial derivative, a disaccharide, a surfactant and an antioxidant. The composition may comprise a pharmacologically acceptable excipient. As used herein “pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient or permitted to dilute or enclose the active ingredient and can be a solid, semi-solid, or liquid agent. It is also envisioned that a pharmaceutical composition comprising a Clostridial derivative can include one or more pharmaceutically acceptable excipients that facilitate processing of an active ingredient into pharmaceutically acceptable compositions. Insofar as any pharmacologically acceptable excipient is not incompatible with the Clostridial derivative, its use in pharmaceutically acceptable compositions is contemplated. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman’s The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

The constituent ingredients of a pharmaceutical composition can be included in a single composition (that is, all the constituent ingredients, except for any required reconstitution fluid, are present at the time of initial compounding of the pharmaceutical composition) or as a two-component system, for example a vacuum-dried composition reconstituted with a reconstitution vehicle which can, for example, contain an ingredient not present in the initial compounding of the pharmaceutical composition. A two-component system can provide several benefits, including that of allowing incorporation of ingredients which are not sufficiently compatible for long-term shelf storage with the first component of the two-component system. For example, the reconstitution vehicle may include a preservative which provides sufficient protection against microbial growth for the use period, for example one-week of refrigerated storage, but is not present during the two-year freezer storage period during which time it might degrade the toxin. Other ingredients, which may not be compatible with a botulinum toxin or other ingredients for long periods of time, can be incorporated in this manner; that is, added in a second vehicle (e.g. in the reconstitution vehicle) at the approximate time of use. A pharmaceutical composition can also include preservative agents such as benzyl alcohol, benzoic acid, phenol, parabens and sorbic acid. Pharmaceutical compositions can include, for example, excipients, such as surface active agents; dispersing agents; inert diluents; granulating and disintegrating agents; binding agents; lubricating agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; antioxidants; stabilizing agents; and pharmaceutically acceptable polymeric or hydrophobic materials and other ingredients known in the art and described, for example in Genaro, ed., 1985, Remington’s Pharmaceutical Sciences, Mack Publishing Co., Easton, Pa., which is incorporated herein by reference.

In embodiments, the botulinum toxin can be selected from the group consisting of botulinum toxin types A, B, C, D, E, F, G, H, X and mosaic toxins. In some embodiments, the botulinum toxin is a botulinum toxin type A. The botulinum toxin can be administered in accord with the treatment paradigm to delivery to the patient a total amount of between about 1 unit and about 3,000 units, or between about 2 units and about 2000 units, or between about 5 units and about 1000 units, or between about 10 units and about 500 units, or between about 15 units and about 250 units, or between about 20 units and about 150 units, or between 25 units and about 100 units, or between about 30 units and about 75 units, or between about 35 units and about 50 units. In some embodiments, the Clostridial derivative can be administered at a dose of about 0.001 ng to about 1000 ng, preferably about 0.001 ng to about 500 ng, preferably about 0.01 ng to about 250 ng, preferably about 0.1 ng to about 150 ng, preferably about 1 ng to about 100 ng, preferably about 1 ng to about 10 ng. For example, onabotulinumtoxinA can be administered at a dose of about 1 ng, 2 ng, 3 ng, 4 ng, 5 ng, 6 ng, 7 ng, 8 ng, 9 ng or 10 ng. In some embodiments, the alleviation of the symptoms can persist for between about 1 month and about 5 years.

In one embodiment, the Clostridial derivative is administered in a composition that comprise about 100 Units of Clostridium botulinum type A neurotoxin complex, with 0.5 mg of human serum albumin, and 0.9 mg of sodium chloride in a sterile, vacuum-dried state for reconstitution. In other embodiments, the Clostridial derivative is administered in an animal-protein free composition. In some embodiments, the Clostridial derivative is administered in an animal protein free composition that comprises a surfactant and a sugar. In some embodiments, the Clostridial derivative is administered in an animal protein free composition that comprises a botulinum toxin type A, a poloxamer and trehalose or sucrose. In one embodiment, the Clostridial derivative is administered in a composition that comprise a Clostridium botulinum type A neurotoxin complex, with 8% trehalose, 4% poloxamer 188 and 0.2% methionine in a sterile, vacuum-dried state for reconstitution.

The administration step of the Clostridial derivative, such as botulinum toxin type A, may comprise utilizing a needle inserted through one or more layers of the scalp and in the vicinity and along a cranial suture line of the patient, and then withdrawing the needle gradually along the suture line while at the same time delivering the toxin along the suture line. In some embodiments, more than one injection site is utilized to administer the botulinum toxin, such as a botulinum toxin type A or B, along a suture line. Administration can substantially follow along the length of the suture line, e.g. at or up to about 1.5 cm away, more preferably at or up to about 1.0 cm, most preferably at or up to about 0.5 cm from the suture line referenced and utilized as a guide for botulinum toxin administration in accordance with the instant disclosure. In one embodiment, a syringe fitted with a needle and filled with a composition comprising the Clostridial toxin is provided, and the needle is inserted along a suture line(s)) that is below the skin surface to administer the toxin.

### EXAMPLES

The following non-limiting examples provide those of ordinary skill in the art with specific preferred methods within the scope of embodiments of the present methods and are not intended to limit the scope thereof.

### Example 1

### Treatment with a Clostridial Toxin Inhibits Seizure Progression

Test animals (rats) were randomized into three groups for treatment. The first group of animals received no treatment. The second group was treated with the Clostridial toxin onabotulinumtoxinA into an area of the scalp innervated by the greater and lesser occipital nerves. A dose of 5 Units of onabotulinumtoxinA was administered at five administration sites, with 1 Unit given at each site, 7-12 days prior to induction of seizure. The third group of animals received saline injections into the same areas where onabotulinumtoxinA was injected, also 7-12 days prior to induction of seizure.

Glass pipette electrodes capable of recording local field potential and electrocorticogram from 0 to 3000 Hz were used to identify cortical activity before and during epileptiform seizures induced by topical administration of picrotoxin - a selective GABA blocker. Placement of the electrodes was as shown in FIG. 1B.

The electrocorticogram traces of the occipital cortex and parietal cortex of the animals in the second and third groups, Clostridial toxin treated and saline treated, respectively, just before and after topical application of picrotoxin to the occipital cortex to trigger a focal seizure are shown in FIGS. 2A-2B and 3A-3B. This study provides evidence for prevention of development and progression of seizure from focal to generalized by extracranial injections of onabotulinumtoxinA into areas of the scalp that are innervated by the greater and lesser occipital nerves - both originate in C2 and C3 dorsal root ganglia, respectively.

### Example 2

### Treatment of Post Traumatic Epilepsy with a Clostridial Toxin

A 42-year-old woman is an unrestrained passenger in a head-on motor vehicle collision. She suffers head injury with resultant skull fracture, bifrontal subdural hematoma, left frontal cortical contusion and let frontal intraprenchymal hemorrhage. After three weeks of intensive care, she is transferred to a medical ward, and from there to rehabilitation facility. While she does not have epilepsy, she is at risk for post-traumatic seizures that may develop (with ~20% likelihood) in the next ear.

The patient does not want to take any oral medications. She does not have any seizures following the accident. However, 9 months after the accident, she develops episodes of expressive aphasia that within 60 seconds develop into tonic/clonic (TC) seizures of her right arm and right leg, and then 30 seconds later generalizes to bilateral TC seizures with loss of consciousness. She is admitted to the emergency department for IV antiepileptic medication administration after one such seizure that does not resolve spontaneously. Upon recovery, she has difficulty with expressive language. On continuous monitoring, an EEG shows paroxysmal focal spikes slowing over the left frontal lobe associated with expressive aphasia. She is started on oral medications that have to be changed periodically as they are either not tolerated or do not provide sufficient seizure relief. Her best seizure control occurs on lamotrigine where, notwithstanding that the dose is optimized, she nevertheless has episodes of expressive aphasia which progress to right sided TC spasms and generalize but are brief in duration. These occur approximately every month.

She is treated as follows with onabotulinumtoxinA: 5 Units into each of 4 sites of the frontalis muscle, one injection of 10 U into the vicinity of the frontonasal suture, 7 injections of 5 Units dispersed in the vicinity of the coronal suture (sub-fascial) distributed across the full length of the suture, 6 injections of 5 U into the left temporalis muscle with 2 of the injections in the vicinity of the squamous suture above and anterior to the ear and 1 injection in the vicinity of the posterior squamous suture. There are 3 injections of 5 U sub-fascia distributed across the saggital suture. She has episodes of expressive aphasia that last about 10 seconds, but there is no progression to the right side and no generalization. She stopped the lamotrigine. She continues treatment every 3 to 5 months and has monthly episodes of brief expressive aphasia.

### Example 3

### Treatment with a Clostridial Toxin Reduces Seizure Occurrences

An adult woman has several seizures per week characterized by sudden onset of confusion with difficulty understanding language for approximately 20 seconds before loss of consciousness and convulsions lasting up to 1 minute. Diagnostics (EEG and MRI) reveal that the seizure onset zone is in the region of the left temporo-parietal region, overlapping with the cortical receptive language area. Thus, the patient has a seizure focus that is in a region that cannot be approached by epilepsy surgery. Despite trials of multiple anti-epileptic medications that include levetiracetam, oxcarbazepine and lamotrigine, the seizures persist. This patient thus has epilepsy that is pharmacoresistant and inoperable.

The patient is treated unilaterally on the left temporal-parietal region with a total dose of 40 units of BOTOX®, of which 15 units were administered in the vicinity of the lambdoid suture at three sites, 10 units were administered in the vicinity of the parietomastoid suture at two sites and 15 units were administered in the vicinity of the occipital suture at 3 sites. Within a week of treatment, the patient does not have any seizure. Over the next three months, the patient has only three seizures. During the fourth month, she experiences three seizures and returns for further treatments. She is retreated with the same treatment pattern every three months and remains seizure-free during the treatment period.

### Example 4

### Treatement with a Clostridial Toxin Alleviates a Generalized Seizure

A 4-year-old boy with autism and developmental delay develops frequent brief tonic seizures. His MRI is normal and his EEG reveals frequent slow spike and wave, consistent with Lennox-Gastaut Syndrome. Despite trials of multiple antiepileptic medications and ketogenic diet, the seizures continue.

The patient is treated bilaterally with onabotulinumtoxinA 5 Units per administration site as follows: 3 injections into each temporalis muscle, 2 injections into each occipitalis muscle, 2 injections along the parietomastoid suture on each side, 2 injections into the cervical paraspinal muscles innervated by cervical nerves on each side for a total of 18 injections for a total of 90 Units. Over the next 3 months, the patient experiences a 50% decrease in seizure frequency. The patient is retreated at the end of three months using the same protocol, with additional sites as follows: 4 injections along the coronal suture and 3 injections along the saggital suture, one injection along the posterior squamous suture on each side, and 2 along the lambdoid suture along each side. The additional treatment provided an additional 13 injections, adding 65 units. In total, the treatment was 155 Units. The patient is treated with this paradigm every 3 months and is seizure-free.

### Example 5

### Treatment with a Clostridial Toxin Reduces a Focal Seizure Associated with Brain Tumor

A 35-year old man with glioblastoma multiforme originating in the right hemisphere, but spreading across the corpus callosum to the left, has focal seizures arising independently for the left and right hemisphere. The seizures persist despite trials with multiple antiepileptic medications. However, based on the EEG, the findings were more prominent on the right side.

The patient is treated with onabotulinumtoxinA, 10 U per injection as follows: 4 injections along the coronal suture (2 on each side), 3 injections along the saggital suture, and 3 injections along the right lambdoid suture. The total amount administered is 100 U. Interestingly, the EEG spike activity is markedly suppressed bilaterally and although his EEG continued to be abnormal, he had no clinical seizure activity.

Many alterations and modifications may be made by those having ordinary skill in the art, without departing from the spirit and scope of the disclosure. Therefore, it must be understood that the described embodiments have been set forth only for the purposes of examples, and that the embodiments should not be taken as limiting the scope of the following claims. The following claims are, therefore, to be read to include not only the combination of elements which are literally set forth, but all equivalent elements for performing substantially the same function in substantially the same way to obtain substantially the same result. The claims are thus to be understood to include those that have been described above, those that are conceptually equivalent, and those that incorporate the ideas of the disclosure.

1. A method for treating seizures associated with a seizure disorder or for treating aura seizure symptoms, comprising:
administering to a subject with a seizure disorder or aura seizure symptoms a Clostridial derivative in an amount effective for treating seizures, the Clostridial derivative administered extracranially to at least one administration site.

2. The method of claim 1, wherein administering comprises administering by extramuscular injection, subcutaneous injection, subdermal injection or intradermal injection.

3. The method of claim 1, wherein administering comprises administering by injection to the scalp of the subject.

4. The method of claims 1, wherein administering is to more than one administration site.

5. (canceled)

6. (canceled)

7. (canceled)

8. The method of claim 1, wherein the at least one administration site is to an occipital area of the cranium that is innervated by cervical nerves.

9. (canceled)

10. (canceled)

11. The method of claim 1, wherein the Clostridial derivative is a botulinum neurotoxin.

12. (canceled)

13. The method of claim 11, wherein the botulinum neurotoxin is botulinum toxin type A, botulinum toxin type B, botulinum toxin type C, botulinum toxin type D, or botulinum toxin type E.

14. The method of claim 11, wherein the botulinum toxin is selected from the group consisting of onabotulinumtoxinA, abobotulinumtoxinA, incobotulinumtoxinA, daxibotulinumtoxinA, prabotulinumtoxinA, and rimabotulinumtoxinB.

15. The method of claim 13, wherein the botulinum toxin comprises a botulinum neurotoxin complex or a pure toxin.

16. (canceled)

17. The method of claim 1, wherein the seizure is a focal onset seizure, a myoclonic seizure, or a primary generalized tonic-clonic seizure.

18. The method of claim 17, wherein the seizures are associated with a seizure disorder selected from epilepsy, juvenile myoclonic epilepsy, idiopathic generalized epilepsy, and Lennox-Gastaut syndrome.

19. (canceled)

20. (canceled)

21. A method for inhibiting progression of a focal seizure to a generalized seizure, comprising:
administering extracranially to a scalp of a subject afflicted by focal seizures a Clostridial derivative in a therapeutically effective amount to inhibit progression of a focal seizure to a generalized seizure.

22. (canceled)

23. A method for treating seizures in a subject with epilepsy or for treating aura seizure (or epilepsy aura) symptoms in a subject with aura seizure (or epilepsy aura) symptoms, comprising:
administering extracranially to the scalp of the subject a Clostridial derivative in a therapeutically effective amount for treating the seizures.

24. (canceled)

25. (canceled)

26. The method of claims 21, wherein administering comprises administering by extramuscular injection, subcutaneous injection, subdermal injection or intradermal injection.

27. The method of claims 21, wherein administering comprises administering by injection to the scalp of the subject.

28. (canceled)

29. (canceled)

30. (canceled)

31. (canceled)

32. The method of claim 21, wherein the at least one administration site is to an occipital area of the cranium that is innervated by cervical nerves.

33. (canceled)

34. (canceled)

35. The method of claim 21, wherein the Clostridial derivative is a botulinum neurotoxin.

36. (canceled)

37. The method of claim 35, wherein the botulinum neurotoxin is botulinum toxin type A, botulinum toxin type B, botulinum toxin type C, botulinum toxin type D, or botulinum toxin type E.

38. The method of claim 35, wherein the botulinum toxin is selected from the group consisting of onabotulinumtoxinA, abobotulinumtoxinA, incobotulinumtoxinA, daxibotulinumtoxinA, prabotulinumtoxinA, and rimabotulinumtoxinB.

39. The method of claim 36, wherein the botulinum toxin comprises a botulinum neurotoxin complex or a pure toxin.

40. (canceled)

41. (canceled)
